(19) United States
(12) Patent Application Publication
(10) Pub. No.: US 2011/0230552 A1
(43) Pub. Date: Sep. 22, 2011

(54) USE OF DRONEDARONE FOR THE PREPARATION OF A MEDICAMENT FOR THE PREVENTION OF STROKE OR TRANSIENT ISCHEMIC ATTACK

(75) Inventors: Christophe GAUDIN, Saint Cloud (FR); Nacéra HAMDANI, Colombes (FR); Davide RADZIK, Paris (FR); Martin VAN EICKELS, Berlin (DE)

(73) Assignee: SANOFI-AVENTIS, Paris (FR)

(21) Appl. No.: 13/020,889

(22) Filed: Feb. 4, 2011

Related U.S. Application Data
(63) Continuation of application No. PCT/IB2009/006831, filed on Aug. 3, 2009.
(60) Provisional application No. 61/087,803, filed on Aug. 11, 2008.

(30) Foreign Application Priority Data
Aug. 7, 2008 (EP) 08290761.9

(51) Int. Cl. A61K 31/343; A61P 7/00

(52) U.S. Cl. 514/469

(57) ABSTRACT

Use of dronedarone for the preparation of a medicament for the prevention of stroke or transient ischemic attack.

The instant invention relates to the use of dronedarone for the preparation of a medicament for the prevention of stroke or transient ischemic attack.

2-n-butyl-3-[4-(3-di-n-butylaminopropoxy)benzoyl]-5-méthylsulfonamido-benzofuranne or dronedarone and its pharmaceutically acceptable salts are described in European patent EP 0 471 609 B1.

Dronedarone is a multi-channel blocker that affects calcium, potassium and sodium channels and has anti-adrenergic properties.

Dronedarone is an anti-arrhythmic agent for the treatment of patients with a history of atrial fibrillation or atrial flutter.

Atrial fibrillation (AF) affects about 2.3 million people in North America and 4.5 million people in the European Union and is emerging as a growing public health concern because of the aging of the population

AF is a condition in which the upper chambers of the heart beat in an uncoordinated and disorganized fashion, resulting in a very irregular and fast rhythm (i.e., an irregularly, irregular heartbeat). When blood is not completely pumped out of the heart's chambers, it can pool and clot. If a blood clot forms in the atria, exits the heart and blocks an artery in the brain, a stroke results. Consequently, about 15 percent of strokes result from AF. But stroke can also complicate other conditions like for example hypertension. Also hemorrhagic strokes can be a complication of treatment with an anticoagulant prescribed to prevent the formation of thrombi in particular in patients with AF.

A transient ischemic attack (TIA) is caused by the transient disturbance of blood supply to an area of the brain, resulting in brief neurologic dysfunction that persists usually for less than 1 hour sometimes up to 24 hours; if symptoms persist for a longer time then it is categorized as a stroke.

Transient ischemic attacks are often considered as a warning for an approaching stroke. About one third of patients with transient ischemic attack will have recurrent transient ischemic attacks and another third a stroke due to permanent nerve cell loss.

The most common cause of a transient ischemic attack is an embolus (blood clot) that occludes an artery in the brain. This can come from an atherosclerotic plaque in one of the two carotid arteries or from the heart for example in case of atrial fibrillation.

The most frequent symptoms include temporary amaurosis (loss of vision); aphasia (difficulty speaking); hemiparesis (weakness of one side of the body; paresthesia (numbness), of on one side of the body.

AF is increasingly frequent with advancing age and is often caused by age-related changes in the heart, physical or psychological stress, agents that stimulate the heart, such as caffeine, or as a result of cardiovascular disease. The number is expected to double in the next 20 years. Without appropriate management, AF can lead to serious complications, such as stroke and congestive heart failure.

As most of the studies did not assess the complications associated with atrial fibrillation such as stroke, so the effect of antiarrhythmic drugs on these endpoints is unknown (Cochrane Collaboration, The Cochrane Library, 2008, 2).

In addition, two large studies including antiarrhythmic drugs in AF patients, AFFIRM (D. G. Wyse and al., The New England Journal of Medecine, 2002, vol. 347, p. 1825-1833) and AF-CHF (D. Roy and al., The New England Journal of Medecine, 2008, vol. 358, p. 2667-2677), did not show a significant difference in stroke rates between the rate and rhythm groups (the recommended antiarrhythmic drug in the rhythm group was mainly amiodarone).

Thromboembolic events including strokes are major complications in patients with atrial fibrillation. The etiology of these thromboembolic events are not fully understood. According to the main hypothesis atrial fibrillation leads to blood stasis in the atria, which promotes the formation of blood clots and thereby causes thromboembolic events like stroke if the blood clots reach the systemic circulation. Therefore it was thought that prevention of atrial fibrillation or anticoagulation would prevent thromboembolic events and strokes. Numerous clinical studies have confirmed that proper anticoagulation can prevent thromboembolic events including strokes (Fuster et al.). But, all randomized clinical trials using anti-arrhythmic drugs did not show a reduction in the incidence of stroke, despite effectively maintaining sinus rhythm in the rhythm control or treatment group.

For example, in the AFFIRM trial, Wyse et al. compared a rhythm control (63% amiodarone and 41% sotalol being the most commonly used anti-arrhythmic drugs) to a rate control strategy. As shown in Table 3 of the article by Wyse et al the incidence of stroke or TIA was similar in the rhythm control group (80/2033) compared to the rate control group (77/2027), despite a higher number of patients in the rhythm control group (63%) being in sinus rhythm after 5 years compared to the rate control group (35%).

In the STAF trial, Carlsson el al. compared a rhythm control (42% amiodarone) to a rate control strategy. As shown in Table 2 of the article by Carlsson et al the incidence of stroke or TIA was numerically higher in the rhythm control group (5/100) compared to the rate control group (1/100), despite a highly significant 29% absolute increase in patients with sinus rhythm at the end of the study in the rhythm control group compared to the rate control group.

In the HOT CAFÉ, Opolski et al compared a rate and a rhythm control strategy. As shown in table 2 of the article of Opolski et al 3/104 patients suffered from a stroke during the follow-up in the rhythm control group compared to 0/101 in the rate control group.

In the J-RHYTHM trial, Ogawa et al. compared a rhythm control (85% of patients were on class I anti-arrhythmic drugs) to a rate control strategy. As shown in Table 3 of the article by Ogawa et al the incidence of symptomatic stroke was similar in the rhythm control group (9/419) compared to the rate control group (11/404), despite a highly significant 29% absolute increase in patients with sinus rhythm at 3 years in the rhythm control group compared to the rate control group.

In the SAFE-T trial, Singh et al compared amiodarone, sotalol and placebo in the treatment of patients with persistent atrial fibrillation. Amiodarone and sotalol were both significantly more effective than placebo in increasing the time to a recurrence of atrial fibrillation (a widely used measure for rhythm control) (P<0.001). Amiodarone was six times as effective as sotalol in the intention-to-treat analysis (P<0.001) and four times as effective in the analysis according to the treatment actually received (P<0.001). Despite this effective rhythm control the number of strokes per 100 patient-years of follow-up were similar in all groups for amiodarone: 2.06 major, sotalol: 2.71, and placebo: 1.91 with the lowest rate observed in the placebo group, which had the highest rate of recurrence of atrial fibrillation (calculated from bottom of last paragraph on page 1866)).

Therefore, administering a drug for preventing atrial fibrillation cannot be considered as implying a prevention of stroke, according to the current knowledge in the Art. Unexpectedly, dronedarone has demonstrated, in the ATHENA trial (Hohnloser et al.), its ability to reduce the incidence of stroke. The effect now seen with dronedarone is not based upon rhythm control alone but on the unique combination of properties of dronedarone, which include but are not limited to: effective rhythm control, heart rate lowering effects, blood pressure lowering effects, direct effects on the endothelial function and others.

The Inventors have now clinically proven that dronedarone reduces the occurrence of stroke while this was not demonstrated for other antiarrhythmic compounds.

The subject of the instant invention is the use of dronedarone or one of its pharmaceutically acceptable salts for the preparation of a medicament for the prevention of stroke or transient ischemic attack notably in patients with a history of atrial fibrillation or atrial flutter.

The subject of the instant invention is also the use of dronedarone or one of its pharmaceutically acceptable salts for the preparation of a medicament for the prevention of stroke notably in patients with a history of atrial fibrillation or atrial flutter.

In contrast to cerebral circulatory insufficiency, which is a chronic disease with slowly deteriorating cognitive function a stroke is an acutely or subacutely evolving neurological deficit of cerebrovascular cause defined by symptoms that persists beyond 24 hours due to a disturbance in the blood vessels of the brain or defined by imaging of an acute clinically relevant brain lesion in patients with rapidly vanishing symptoms.

This can be due to ischemia (lack of blood supply) caused by thrombosis or embolism, or due to a haemorrhage. (R. L. Sacco et al., Stroke, 2006; vol. 37 p. 577-617)

Stroke can cause permanent neurological damage or death. It is the leading cause of adult disability in the United States and Europe.

Risk factors for stroke include advanced age, hypertension, previous stroke or transient ischemic attack (TIA), diabetes, high cholesterol, cigarette smoking, atrial fibrillation, etc. Hypertension is the most important modifiable risk factor of stroke.

The symptoms of a stroke are similar to those of a transient ischemic attack but last more than 24 hours.

The main strokes are ischemic or hemorrhagic strokes. Ischemic strokes are more frequent and in some case could become hemorrhagic strokes.

In an embodiment, the invention relates to the use of dronedarone or one of its pharmaceutically acceptable salts for the preparation of a medicament for the prevention of ischemic stroke notably in patients with a history of atrial fibrillation or atrial flutter.

More precisely, the invention relates to the use of dronedarone or one of its pharmaceutically acceptable salts for the preparation of a medicament for the prevention of about 35% of stroke or transient ischemic attack in patients with a history of atrial fibrillation or atrial flutter.

More precisely, the invention relates to the use of dronedarone or one of its pharmaceutically acceptable salts for the preparation of a medicament for the prevention of about 35% of stroke in patients with a history of atrial fibrillation or atrial flutter.

In another embodiment, the invention relates to the use of dronedarone or one of its pharmaceutically acceptable salts for the preparation of a medicament for the prevention of fatal stroke.

A fatal stroke is defined as a stroke leading to death.

In another embodiment, the invention relates to the use of dronedarone or one of its pharmaceutically acceptable salts for the preparation of a medicament for the prevention of stroke, acute coronary syndrome and death or cardiovascular death.

In another embodiment, the invention relates to the use of dronedarone or one of its pharmaceutically acceptable salts for the preparation of a medicament for the prevention of acute coronary syndrome (ACS).

The composite endpoint of stroke, acute coronary syndrome and death or cardiovascular death is a classical outcome measure in cardiovascular outcomes trials also called MACE (major adverse cardiovascular events) endpoint. The inclusion of this endpoint, shows the broader impact and relevance of the finding on stroke.

The data on ACS alone are connected to stroke, by the fact that both are ischemic events.

In terms of clinical study, the prevention of “stroke, acute coronary syndrome and death or cardiovascular death” constitute what are referred to as composite criteria or a combined endpoint.

The percentages above correspond to an average.

Among the pharmaceutically acceptable salts of dronedarone, mention may be made of the hydrochloride.

The treated patients may be patients with a history of atrial fibrillation or atrial flutter.

The expression <<with a history of atrial fibrillation or atrial flutter>> means a patient who has previously manifested at least one symptom of atrial fibrillation (AF) or atrial flutter (AFL) and who can be either in sinus rhythm or in atrial fibrillation or atrial flutter at the time of dronedarone administration.

It will also be specified that the expression “patients having a history of atrial fibrillation or atrial flutter”, “patients with a history of or a current atrial fibrillation or flutter” or “patients with a recent history of or a current atrial fibrillation or flutter” or “patients with paroxysmal or persistent atrial fibrillation or flutter” or “patients with a history of, or a current paroxysmal or persistent atrial fibrillation or flutter” or “patients with a recent history of, or a current paroxysmal or persistent atrial fibrillation or flutter” or “patients with paroxysmal or intermittent atrial fibrillation or atrial flutter and a recent episode of atrial fibrillation or atrial flutter, who are in sinus rhythm or who will be cardioverted” or “patients with paroxysmal or persistent atrial fibrillation or atrial flutter and a recent episode of atrial fibrillation or atrial flutter, who are in sinus rhythm or who will be cardioverted” means a patient who, in the past, has presented one or more episodes of atrial fibrillation or flutter and/or who is suffering from atrial fibrillation or atrial flutter at the time the dronedarone or a pharmaceutically acceptable salt thereof is used. More particularly, this expression means patients with documentation of having been in both atrial fibrillation or flutter and sinus rhythm within the last 6 months preceding the start of treatment. Patients could be either in sinus rhythm, or in atrial fibrillation or flutter at the time the dronedarone or a pharmaceutically acceptable salt thereof is initiated.

It will also be specified that the terms “persistent” and intermittent” are equivalent.

Patients in “permanent atrial fibrillation or flutter” are patients that have all scheduled ECGs in this rhythm throughout the period the dronedarone or a pharmaceutically acceptable salt thereof is administered.

Among the patients with a recent history of, or a current atrial fibrillation or atrial flutter, mention may be made of patients with a recent history of, or a current, non permanent atrial fibrillation or flutter.

In the instant invention, “atrial fibrillation” means atrial fibrillation and/or atrial flutter.

Among patients with a history of atrial fibrillation or atrial flutter, mention may be made of patients who further have at least one of the following risk factors:
age, notably equal to or above 70, or even above 75,
hypertension,
diabete,
prior cerebrovascular accident or systemic embolism,
left atrium diameter greater that or equal to 50 mm by echocardiography,
left ventricular ejection fraction less than 40% by 2D-echocardiography.

Among patients with a history of atrial fibrillation or atrial flutter, mention may also be made of patients having additional risk factors corresponding to at least one of the following diseases:
hypertension,
structural heart disease,
tachycardia,
coronary heart disease,
non-rheumatic valvular heart disease,
ischemic dilated cardiomyopathy,
a history of ablation for AF/AFL for example catheter ablation or surgical ablation,
supra-ventricular tachycardia other than AF/AFL,
history of cardiac valve surgery,
non-ischemic dilated cardiomyopathy,
hypertrophic cardiomyopathy,
rheumatic valvular heart disease,
sustained ventricular tachycardia,
congenital heart disease,
a history of ablation for other reason than AF/AFL for example catheter ablation,
ventricular fibrillation,
   and/or at least a cardiac device chosen among:
a pacemaker,
an implanted cardioverter defibrillator.

Mention may be made that congestive heart failure is a sub-group of structural heart disease.

Another object of the invention is a pharmaceutical composition which comprises, as active principle, dronedarone or one of its pharmaceutically acceptable salts. This pharmaceutical composition comprises an effective dose of at least one compound of formula (I) according to the invention, or an addition salt thereof with a pharmaceutically acceptable salt, or a hydrate or solvate thereof, and at least one pharmaceutically acceptable excipient. Said excipients are chosen according to the pharmaceutical form and the administration route desired, among usual excipients known to one of skill in the art.

In the pharmaceutical compositions according to the invention for the oral, sublingual, sub-cutaneous, intramuscular, intra-venous, topical, local, intratracheal, intranasal, transdermal or rectal administration, dronedarone or one of its salt, solvate or hydrate, can be administered as a unitary dosage form, in blend with usual pharmaceutical excipients, to animals and human beings for the prevention or for the treatment of pathological states mentioned above. The appropriate unitary dosage forms comprise the oral forms, such as tablets, hard or soft gelatin capsules, powders, granules and oral solutions or suspensions, the sublingual, buccal, intratracheal, intraocular, intranasal forms, the forms adapted to inhalation, topical, transdermal, sub-cutaneous, intramuscular or intra-venous delivery, the rectal forms and the implants. For the topical application, the compounds of the invention may be used as creams, gels, ointments or lotions.

For its use in therapeutics, dronedarone and its pharmaceutically acceptable salts are incorporated in pharmaceuticals compositions.

These pharmaceutical compositions comprise an effective dose of at least dronedarone or one of its pharmaceutically acceptable salts and at least one pharmaceutically acceptable excipient.

Said excipients are chosen according to the pharmaceutical form and the administration route desired, among usual excipients known of one of skill in the art.

In the pharmaceutical compositions for the oral, sublingual, sub-cutaneous, intramuscular, intra-venous, topical, local, intratracheal, intranasal, transdermal or rectal administration, dronedarone or one of its pharmaceutically acceptable salts, can be administered as a unitary dosage form, in blend with usual pharmaceutical excipients, to animals and human in diseases above mentioned.

The appropriate unitary dosage forms comprise the oral forms, such as tablets, hard or soft gelatin capsules, powders, granules and oral solutions or suspensions, the sublingual, buccal, intratracheal, intraocular, intranasal forms, by inhalation, the topical, transdermal, sub-cutaneous, intramuscular or intra-venous forms, the rectal forms and the implants. For the topical application, the compounds of the invention may be used as creams, gels, ointments or lotions.

As an example, a unitary dosage form for dronedarone or one of its pharmaceutically acceptable salts, in the form of a tablet, can comprise the following ingredients:

| Ingredients | mg |
|---|---|
| dronedarone hydrochloride (corresponding to | 426 |
| 400 mg of base) | |
| Methylhydroxypropylcellulose | 21.1 |
| Lactose monohydrate | 46.55 |
| Modified corn starch | 45.5 |
| Polyvinylpyrrolidone | 65 |
| Poloxamer 407 | 40 |
| Anhydrous colloidal silica | 2.6 |
| magnesium stearate | 3.25 |
| | 650 |
| dronedarone hydrochloride (corresponding to | 426 |
| 400 mg of base) | |
| microcristalline cellulose | 65 |
| Anhydrous colloidal silica | 2.6 |
| anhydrous lactose | 42.65 |
| Polyvinylpyrrolidone | 13 |
| Poloxamer 407 | 40 |
| Macrogol 6000 | 57.5 |
| magnesium stearate | 3.25 |
| | 650 |
| dronedarone hydrochloride (corresponding to | 426 |
| 400 mg of base) | |
| microcristalline cellulose | 26 |
| corn starch | 45.5 |
| Polyvinylpyrrolidone | 65 |
| Poloxamer 407 | 40 |
| Anhydrous colloidal silica | 3.25 |
| magnesium stearate | 3.25 |
| Lactose monohydrate | 41.65 |
| | 650 |
| dronedarone hydrochloride (corresponding to | 213 |
| 400 mg of base) | |
| microcrystalline cellulose | 13 |
| corn starch | 22.75 |
| Polyvinylpyrrolidone | 32.5 |
| Poloxamer 407 | 20 |
| Anhydrous colloidal silica | 1.3 |
| magnesium stearate | 1.625 |
| Lactose monohydrate | 20.825 |
| | 650 |

Said pharmaceutical composition may be given once or twice a day with food.

The dose of dronedarone administered per day, orally, may reach 800 mg, taken in one or more intakes, for example one or two.

More specifically, the dose of dronedarone administered may be taken with food.

More specifically, the dose of dronedarone administered per day, orally, may reach 800 mg, taken in two intakes with a meal.

The dose of dronedarone administered per day, orally may be taken at a rate of twice a day with a meal for example with the morning and the evening meal.

More specifically, the two intakes may comprise same quantity of dronedarone.

In specific cases, higher or lower dosages may be appropriate; these dosages are comprised within the scope of the present invention. According to usual practice, the dosage suitable to each patient is determined by the physician according to the administration route, the weight, the disease, the body surface, the cardiac output and response of the patient.

The instant invention also relates to a method of prevention of stroke which comprises the administration to a patient of an effective dose of at least dronedarone or one of its pharmaceutically acceptable salts.

The invention is illustrated with the above data with reference to the following figure:

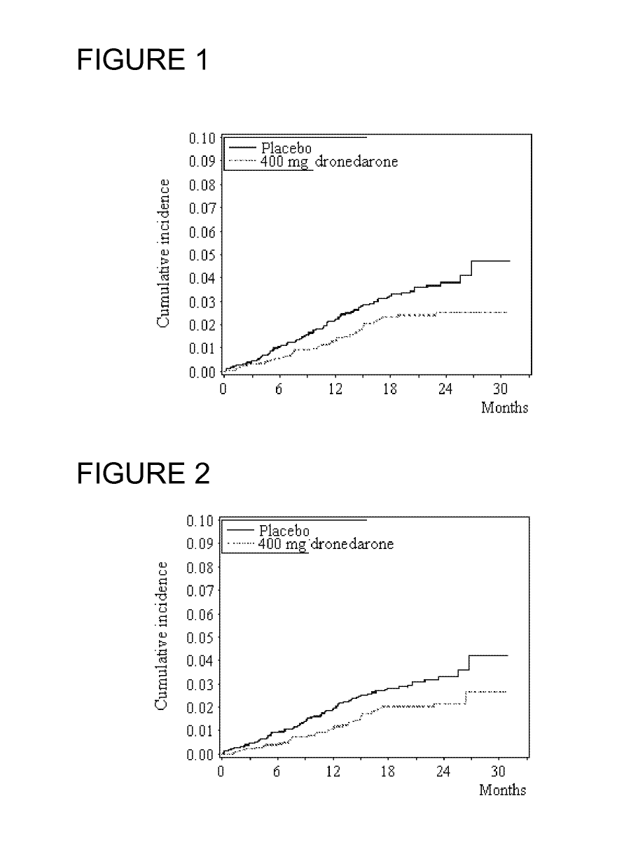

FIG. 1 represents Kaplan Meier cumulative incidence curves of time to first stroke or TIA according to the on-treatment analysis of 30 months;

FIG. 2 represents Kaplan Meier cumulative incidence curves of time to first stroke according to the on-treatment analysis of 30 months.

Efficacy of dronedarone and its pharmaceutically acceptable salts versus placebo for the prevention of stroke was provided via dronedarone hydrochloride during a prospective, multinational, double-blind, randomized, multi-center, placebo-controlled, parallel group trial.

### I. Selection of Patients

Eligible patients have a history of atrial fibrillation or atrial flutter and/or may be in normal sinus rhythm or in atrial fibrillation or flutter at the time of recruitment.
Recruitment of patients was conducted taking into account the following inclusion criteria:

### Inclusion Criteria:

1) One of the following risk factors had to be present:
age equal to or greater than 70 years,
hypertension (taking antihypertensives of at least two different classes),
diabetes,
history of cerebral stroke (transient ischemic event or completed cerebral stroke) or of systemic embolism,
left atrial diameter greater than or equal to 50 mm measured by echocardiography,
left ventricular ejection fraction less than 40%, measured by two-dimensional echography;
   or
age equal to or above 70, or even above 75, possibly combined with at least one of the risk factors below:
   hypertension (taking antihypertensives of at least two different classes),
   diabetes,
   history of cerebral stroke (transient ischemic event or completed cerebral stroke) or of systemic embolism,
   left atrial diameter greater than or equal to 50 mm measured by echocardiography,
   left ventricular ejection fraction less than 40%, measured by two-dimensional echography;

2) availability of one electrocardiogram within the last six months, showing that the patients was or is in atrial fibrillation/flutter,

3) availability of one electrocardiogram within the last six months, showing that the patients was or is in sinus rhythm.

### II. Duration and Treatment

Study drug treatment units (placebo or dronedarone hydrochloride corresponding to 400 mg of base) were such that each patient took one tablet in the morning during or shortly after breakfast and one tablet in the evening during or shortly after dinner.

The treatment duration depended on the time of recruitment of each patient in the trial and could be comprised from 12 months to 30 months.

### III. Results

Results were calculated using non-parametric Kaplan-Meier estimate.

Cox's proportional hazard model was used to estimate the hazard ratio also called relative risk.

Relative risk (RR) is the ratio between the risk of having a stroke (or transient ischemic attack (TIA)) for patients treated with dronedarone and the risk of having a stroke (or transient ischemic attack (TIA)) for patients treated with placebo.

Percentage of decrease of an event is calculated as follow:

x=1−RR.

### Results Relating to the Prevention of Stroke or Transient Ischemic Attack (TIA)

From the 4628 patients included in the trial, 2301 were part of the group treated with dronedarone hydrochloride.
80 stroke or TIA events were reported in the placebo group versus 52 in the group treated with dronedarone hydrochloride.
Calculated relative risk was equal to 0.65, i.e. a decrease of the relative risk of stroke or TIA of 35%.
FIG. 1 shows that the effect of dronedarone occurred early and increased over time.

### Results Relating to the Prevention of Stroke

From the 4628 patients included in the trial, 2301 were part of the group treated with dronedarone hydrochloride.
70 stroke events were reported in the placebo group versus 45 in the group treated with dronedarone hydrochloride.
Calculated relative risk was equal to 0.65, i.e. a decrease of the relative risk of stroke of 35%.
FIG. 2 shows that the effect of dronedarone occurred early and increased over time.

### Results Relating to the Prevention of Ischemic Stroke

From the 4628 patients included in the trial, 2301 were part of the group treated with dronedarone hydrochloride.
49 stroke events were reported in the placebo group versus 33 in the group treated with dronedarone hydrochloride.
Calculated relative risk was equal to 0.68, i.e. a decrease of the relative risk of ischemic stroke of 32%.

### Results Relating to the Prevention of Fatal Stroke

From the 4628 patients included in the trial, 2301 were part of the group treated with dronedarone hydrochloride.
18 fatal stroke events were reported in the placebo group versus 11 in the group treated with dronedarone hydrochloride.
Calculated relative risk was equal to 0.62, i.e. a decrease of the relative risk of fatal stroke of 38%.

### Results Relating to the Prevention of Stroke, Acute Coronary Syndrome (Acs) or Death

From the 4628 patients included in the trial, 2301 were part of the group treated with dronedarone hydrochloride.
262 events were reported in the placebo group versus 196 in the group treated with dronedarone hydrochloride.
Calculated relative risk was equal to 0.68, i.e. a decrease of the relative risk of stroke, Acute Coronary Syndrome (ACS) or death of 25%.

### Results Relating to the Prevention of Stroke, Acute Coronary Syndrome (ACS) or Cardiovascular Death

From the 4628 patients included in the trial, 2301 were part of the group treated with dronedarone hydrochloride.
216 events were reported in the placebo group versus 147 in the group treated with dronedarone hydrochloride.
Calculated relative risk was equal to 0.68, i.e. a decrease of the relative risk of stroke, Acute Coronary Syndrome (ACS) or cardiovascular death of 32%.

### Results Relating to the Prevention of Cardiovascular Hospitalization for Acute Coronary Syndrome (ACS)

From the 4628 patients included in the trial, 2301 were part of the group treated with dronedarone hydrochloride.
89 ACS events were reported in the placebo group versus 62 in the group treated with dronedarone hydrochloride.
Calculated relative risk was equal to 0.70, i.e. a decrease of the relative risk of cardiovascular hospitalization for Acute Coronary Syndrome of 30%.
Results Relating to the Prevention of Stroke for Patient with Additional Risk Factors Such CHADS2 Score, CHF, Hypertension, Age, Diabete Mellitus, Previous Stroke or TIA

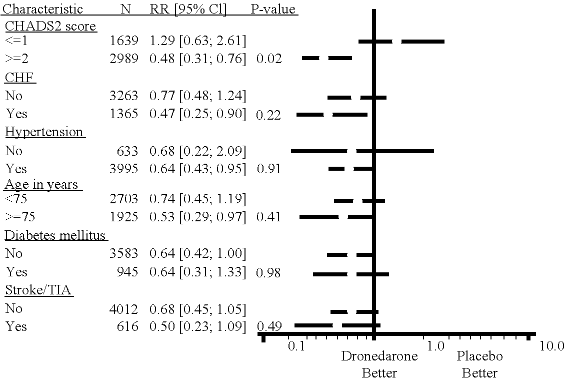

The CHADS2 score, which is calculated by assigning 1 point each for the presence of congestive heart failure, hypertension, age 75 years or older, and diabetes mellitus and by assigning 2 points for history of stroke or TIA characterizes the risk of stroke in patients with AF. The higher the CHADS2 score the higher the risk of stroke. Gage B F, van Walraven C, Pearce L, Hart R G, Koudstaal P J, Boode B S, Petersen P. Selecting patients with atrial fibrillation for anticoagulation: stroke risk stratification in patients taking aspirin. Circulation 2004; 110:2287-92.

We claim:

1. A method for preventing stroke or transient ischemic attack, in a patient in need thereof, comprising administering to the patient an effective dose of dronedarone.

2. The method according to claim 1 for preventing stroke.

3. The method according to claim 1 for preventing about 35% of stroke.

4. The method according to claim 1, wherein the patient has a history of, or a current atrial fibrillation or atrial flutter.

5. The method according to claim 1, wherein the patient has at least one of the following risk factors:
age,
hypertension,
diabete,
prior cerebrovascular accident or systemic embolism,
left atrium diameter greater that or equal to 50 mm by echocardiography,
left ventricular ejection fraction less than 40% by 2D-echocardiography.

6. The method according to claim 1, wherein the patient has additional risk factors corresponding to at least one of the following diseases:
hypertension,
structural heart disease,
tachycardia,
coronary heart disease,
non-rheumatic valvular heart disease,
ischemic dilated cardiomyopathy,
ablation for AF/AFL,
supra-ventricular tachycardia other than AF/AFL,
history of cardiac valve surgery,
non-ischemic dilated cardiomyopathy,
hypertrophic cardiomyopathy,
rheumatic valvular heart disease,
sustained ventricular tachycardia,
congenital heart disease,
ablation for other reason than AF/AFL,
ventricular fibrillation,
or at least a cardiac device chosen among:
a pacemaker,
an implanted cardioverter defibrillator.

7. The method according to claim 1, wherein for oral administration, dronedarone daily dose may reach 800 mg.

8. A method for preventing acute coronary syndrome, in a patient in need thereof, comprising administering to the patient an effective dose of dronedarone.

9. The method according to claim 8, wherein the patient has a history of, or a current atrial fibrillation or atrial flutter.

10. The method according to claim 8, wherein the patient has at least one of the following risk factors:
age,
hypertension,
diabete,
prior cerebrovascular accident or systemic embolism,
left atrium diameter greater that or equal to 50 mm by echocardiography,
left ventricular ejection fraction less than 40% by 2D-echocardiography.

11. The method according to claim 7, wherein the patient has additional risk factors corresponding to at least one of the following diseases:
hypertension,
structural heart disease,
tachycardia,
coronary heart disease,
non-rheumatic valvular heart disease,
ischemic dilated cardiomyopathy,
ablation for AF/AFL,
supra-ventricular tachycardia other than AF/AFL,
history of cardiac valve surgery,
non-ischemic dilated cardiomyopathy,
hypertrophic cardiomyopathy,
rheumatic valvular heart disease,
sustained ventricular tachycardia,
congenital heart disease,
ablation for other reason than AF/AFL,
ventricular fibrillation,
or at least a cardiac device chosen among:
a pacemaker,
an implanted cardioverter defibrillator.

12. A method for preventing fatal stroke, in a patient in need thereof, comprising administering to the patient an effective dose of dronedarone.

13. The method according to claim 12, wherein the patient has a history of, or a current atrial fibrillation or atrial flutter.

14. The method according to claim 12, wherein the patient has at least one of the following risk factors:
age,
hypertension,
diabete,
prior cerebrovascular accident or systemic embolism,
left atrium diameter greater that or equal to 50 mm by echocardiography,
left ventricular ejection fraction less than 40% by 2D-echocardiography.

15. The method according to claim 12, wherein the patient has additional risk factors corresponding to at least one of the following diseases:
hypertension,
structural heart disease,
tachycardia,
coronary heart disease,
non-rheumatic valvular heart disease,
ischemic dilated cardiomyopathy,
ablation for AF/AFL,
supra-ventricular tachycardia other than AF/AFL,
history of cardiac valve surgery,
non-ischemic dilated cardiomyopathy,
hypertrophic cardiomyopathy,
rheumatic valvular heart disease,
sustained ventricular tachycardia,
congenital heart disease,
ablation for other reason than AF/AFL,
ventricular fibrillation,
or at least a cardiac device chosen among:
a pacemaker,
an implanted cardioverter defibrillator.

16. The method according to claim 12, wherein for oral administration, dronedarone daily dose may reach 800 mg.

17. A method for preventing stroke, in a patient in need thereof, comprising administering to the patient an effective dose of dronedarone or one of its pharmaceutically acceptable salts.
